(19) United States
(12) Patent Application Publication
(10) Pub. No.: US 2023/0228705 A1
(43) Pub. Date: Jul. 20, 2023

(54) PROCESSES AND METHODS FOR FABRICATION AND USE OF CARBON NANOTUBULE AND GRAPHENE MATRICES

(71) Applicant: Richard Postrel, Miami Beach, FL (US)

(72) Inventor: Richard Postrel, Miami Beach, FL (US)

(21) Appl. No.: 17/676,898

(22) Filed: Feb. 22, 2022

Related U.S. Application Data
(63) Continuation of application No. PCT/US2022/013206, filed on Jan. 20, 2022.

(51) Int. Cl. G01N 27/414; B82Y 15/00; B82Y 30/00; C01B 32/159; G16B 35/00; G16B 40/00; G01N 27/327

(52) U.S. Cl. CPC G01N 27/4146; B82Y 15/00; B82Y 30/00; C01B 32/159; G16B 35/00; G16B 40/00; G01N 27/3278

(57) ABSTRACT

The present invention teaches multiple three-dimensional nanosensing geometries for simultaneously assaying both large and small bio-related molecules in one device. The invention delivers broader sensitivity and selectivity than devices that assay small or large molecules separately. The combination assays all classes of molecules, e.g., proteins, lipoproteins, nucleoproteins, lipids, phospholipids, carbohydrates, nucleic acids, simple sugars, hormones, volatile organic compounds, drugs, drug metabolites, etc. Broad collection enables i) rapid and accurate diagnosis, ii) likely courses of treatments, and iii) timely feedback that monitors and follows the progressions of treatment(s). In one example, a patient's pattern of blood lipids, proteins—including proteins with alternate cleavage patterns, peptides—including endocrine peptides, thyroxine (and/or other hormones), and drug metabolites, forms a profile specific to that patient at that time. The profile is inputted for analysis by comparing it to a library of pooled data. Applying artificial intelligence (AI) to this comparison allows accurate diagnosis and then can suggest historically validated treatments most suited to that patient.

The present invention teaches multiple three-dimensional nanosensing geometries for simultaneously assaying both large and small bio-related molecules within a single device or a set of interconnected devices. The matrices of the invention combine to result in broader sensitivity and selectivity from sensors in devices that assay small or large molecules separately. The combination assays all classes of molecules, e.g., proteins, lipoproteins, nucleoproteins, lipids, phospholipids, carbohydrates, nucleic acids, simple sugars, hormones, volatile organic compounds, drugs, drug metabolites, etc. Such broad collection enables i) rapid and accurate diagnosis, ii) likely courses of treatments, and iii) timely feedback that monitors and follows the progressions of treatment(s). In one example, a patient's pattern of blood lipids, proteins—including proteins with alternate cleavage patterns, peptides— including endocrine peptides, thyroxine (and/or other hormones), and drug metabolites, forms a profile specific to that patient at that time. The profile is inputted for analysis by comparing this patient's profile to a library of patient data. Applying artificial intelligence (Al) to this comparison allows accurate diagnosis and then can suggest historically validated treatments most suited to that patient.

This multi-faceted device is capable of recognizing and distinguishing patterns of interacting molecules present in a sample, e.g., graphene based sensors for liquid sample or liquid off-gas and single wall carbon nanotubes (SWNT) based sensors for volatile organic compounds. Both types of sensors function by altering conductivity (or other electronic output) of a sensor element when it encounters the dissolved molecules and/or the volatile organic compounds of the sample. Different compounds in the samples will present with highly specific conductivity charge patterns.

Biochemical reactions, the reactions sustaining life in all living things, produce small molecules that are released or emitted from the internal aqueous environment of living cells and organisms into a surrounding ambient gas volume. Especially preferred are p-type Single Walled carbon NanoTubes (SWNTs) disposed onto a substrate chip. SWNTs may be understood as a form of graphene wherein a single layer of two dimensional carbon forms a tube typically 1 to 2 nm in diameter (aka tubular graphene) They can be up to hundreds of millimeters in length, though for the present invention shorter lengths (micron range) are preferred, for example SWNTs having a length to diameter ratio about 10,000:1, ≤about 5000:1, ≤about 4000:1, ≤about 3000:1, ≤about 2000:1, ≤about 1200:1, ≤about 1000:1, ≤about 800:1, ≤about 700:1, about 600:1, ≤about 500:1, ≤about 400:1, ≤about 300:1, ≤about 200:1, ≤about 100:1, and/or ≤about 10:1. A sensing element may feature a range of lengths interfacing with one another. Length to diameter ratios between about 600:1 and about 1000:1 are preferred for forming robust stacking geometries. A sensing element may include SWNTs with different length to diameter ratios.

Non-tubular graphene may be used in conjunction with SWNTs. For example, a non-tubular, essentially flat graphene layer may serve as a substrate layer atop which one or more layers of SWNTs may be disposed. Two essentially flat graphene layers may form borders separated by SWNTs, somewhat like a sandwich with top and bottom comprising essentially flat graphene and the inner layers comprising SWNTs.

For purposes of this discussion, a sensor element is a construction that provides a single output channel for a construction of carbon sensors, planar of tubular, in contact with one another. The expression “essentially flat” primarily is meant to distinguish from tubular graphene constructs, that is a two dimensional form of graphene with more than two sides from the tubular format with two ends to a hollow tube. Another manner of picturing the difference would be to describe a tubular form as a construct where a line drawn on the surface of the graphene as the graphene is moved in one continuing direction perpendicular to the pen drawing the line can form a continuous closed shape with no end, i.e., returning to the initiation point, or a line with a length that is a multiple of the projected width and length of the graphene layer that extends from one side (end) to an essentially parallel end (side). I.e the SWNT is more like a straw whereas “essentially flat” graphene is more like paper.

In practice, “essentially flat” graphene structures can be formed as crumpled surfaces or as ultra flat-surfaces, while tubular graphene, carbon nanotubes (CNTs), or SWNTs exist as tubes. Carbon nanotubes, especially those formed as SWNTs, can be fabricated and disposed on a substrate to form high surface area scaffolds prepared for the attraction or transient attachment of interacting molecules such as VOCs. Interacting molecules generally transiently or reversibly interact due to weak attractant forces which disrupt the pattern of cloud of electrons of one or more SWNTs or or one or more molecules pre-disposed on (decorating or functionalizing) the sensor element surface. Volatile compounds in close vicinity of a sensor thereby bias electron clouds on the sensor surface and change the electronic effect of the surface sensor molecule. This in turn changes how that molecule electronically interacts with its disposition surface resulting in activation of a Field Effect Transistor (FET) or FET-like effect in associated molecules of the sensor element which service the output electrode(s).

The substrate chip supporting the sensor element is not a liming feature of the present invention. Preference may be given to chips that an individual, wishing to practice this invention, has easy access to, or familiarity with. A silicon oxide (SiO₂)/Si substrate chip is a useful example as a supporting substrate. These are small, easily produced or obtained, and can be configured with desired circuitry, metallic addends, ID tags, antennas, magnetic spots, etc. Such substrate may serve as a primary substrate in support of a secondary, tertiary substrate layer, etc. One such secondary substrate disposable upon the Si based chip substrate is an essentially flat graphene layer. An “essentially flat” graphene substrate may serve as a secondary substrate layer in association with, e.g., a conventional Si-based chip substrate. Graphene has desirable properties including, but not limited to: minimal electron dispersion, high thermal stability, bio-compatibility, transparency, low adhesion, high strength, low porosity, etc. FET circuits may be made, for example with Cr/Au source and drain electrodes. These may be written, for example with electron beam lithography. A degeneratively doped silicon substrate may serve as a backgate.

A backgate may incorporated into a lower portion of a fin with a tri-gate structure electrically isolated from the backgate. The backgate may then be used to control the threshold voltage of a FET. A backgate may extend to an n-well on a p-type silicon substrate. A contact to the n-well applies electrical voltage to the backgate. A diode between the n-well and p-substrate isolates the current flowing through the n-well from other devices on the substrate allowing independent bias of the backgate, Alternatively, the backgate may extend to an n-type poly-silicon layer on an insulator layer disposed on a p-type silicon substrate. In this version, contact to the n-type poly-silicon layer applies electrical voltage to the backgate. A trench isolation structure may be used to isolate current flowing through a poly-silicon layer from other devices disposed on the silicon substrate. U.S. Pat. No. 7,888,743 incorporated in its entirety by reference, evidences previous state of the art and serves as instructive guidance for making and using such circuitry including examples of dopant technology.

Nano FETs operate by changing electrical properties—when a substance comes in close proximity to the sensor—by perturbing the steady state (absent the proximal substance) charges (distribution of electrons) within the nano-sensor. The altered distribution of electrons, depending on the design of the nano-sensor, changes one or more electrical properties, e.g., impedance, resistance-conductivity, capacitance, inductance, etc. and thus the physical movement of a detectable particle, e.g., an electron, a photon emission, etc. The present invention primarily features nano-sensors whose characteristics change depending upon association (close proximity with) a chemical substance. Sensation may involve more than one event. For example, in one format of nano-sensor, the proximity event may dampen a vibration that is sensed by observing a changed electrical property. Similarly, an optical property, e.g., reflectance, transmittance, refractive index, can be perturbed by proximity to a substance, altering electron distribution within the sensor sufficient to cause optically detectable geometric changes. The optically related detection format for a nano-sensor may be observed at a specific frequency or range of frequencies, for example moving peak transmittance to another frequency.

Although ambient air may remain in a sensing compartment, for peak selectivity and senstivity, the sensor elements themselves, or at least portions of the device surrounding the chips, are preferably surrounded by a controlled gaseous atmosphere. The sensing chamber itself may have a reduced pressure with respect to the sample introduction area. A positive device pressure analogous to a “clean room” environment is generally preferred to maintained to minimize contaminating inflows. Thus, in preferred embodiments, after a sample is introduced into a loading zone, the pressure in the loading zone is increased mechanically or by raising the temperature. The increased pressure will prevent inflow through leaks in the enclosure but will serve to deliver a flow of assay gas into the sensing chamber.

The actual pressure where sensing is accomplished however can be at the discretion of the user and can be varied in a single run or in serial runs. VOCs may be delivered by having a relative pressure in the chip area negative with respect to a sample containment or introduction area. This results in the drawing in of sample off-gas when the off-gas collection volume and the sensing volume become connected. Heating alone is one option for increasing pressure. VOCs themselves, for example, when heated may produce or help produce the pressure difference to drive delivery to the sensor volume.

US application, 2017-0209570, hereby incorporated in its entirety by reference, discloses carbon tube structures including specific teachings that: “CNTs that form the scaffold may be either single-walled CNTs (SWNTs) or multi-walled CNTs (MWNTs). In a preferred embodiment, the compositions contain SWNTs. SWNTs are formed by a single graphene layer rolled-up in the shape of a cylinder. MWNTs are formed by two or more graphene layers rolled-up in the shape of a cylinder. Single-walled carbon nanotubes may assume three types of shapes, termed “armchair”, “zigzag”, and “chiral”, depending on how the six-membered rings are arranged.” and “A carbon nanotube is a cylindrical structure in which such a layer is rolled-up in one direction. In general, those with a diameter of several nanometers to several ten of nanometers and a length of several ten times to not less than several thousand times longer than its diameter are called ‘carbon nanotubes’”.

CNTs have been fabricated using several methods including, but not limited to: arc-discharge methods, laser vaporation methods, thermal chemical vapor deposition methods, flowing vapor deposition methods, etc. For example, the arc-discharge method comprises growing CNTs by means of arc discharge from carbon electrodes. This method is capable of producing a tremendous output of CNTs. The laser evaporation method essentially forms CNTs by evaporating molecules off a part of a graphite electrode excited by a laser. The thermal CVD method forms carbon nanotubes at a high temperature by thermally decomposing hydrocarbons used as a carbon source onto a substrate with a predisposed metal catalyst. The flowing vapor deposition method forms carbon nanotubes through an organic transition metal compound and a hydrocarbon compound, transported with a carrier gas, while reacting with one another at a high temperature.

In accordance with the present invention, the sensor element comprises a substrate; an insulating layer interfaced with source and drain electrodes, and at least one nanotube layer interactive with sensed molecules. The nanotube layer in this invention comprises SWNTs in a compounded stacking formation. While a plurality of nanotubes contact the insulating layer, the compounded stacking formation features nanotubes atop nanotubes with a plurality electronically contacting the insulating layer using physical bridging through nanotubes in contact with the insulating layer. The stacking results from SWNTs loosely adhering to neighbor SWNTs. When a first layer is disposed on a substrate, interaction is maximized by the SWNT lying flat on the surface. These SWNTs do not stack in a parallel formation, but rather just lie where the adherence begins, When a nanotube first contacts the substrate, adherence forces draw it into a flattened position, rather independently of other nanotubes. They thus lie in a two dimensional pattern pointing in different directions. When a subsequent layer is applied, an initial contact may draw the nanotube closer to the substrate surface, but the surface is speckled with nanotubes that prevent the later layers of SWNTs from lying flat upon the substrate. As the later arriving nanotubes settled towards the surface, they may be drawn toward and subsequently contact one or more predisposed nanotubes along their length.

Thus a second layer will present with nanotubes in contact with the substrate layer but having one or more ends or one or more internal portions separated from the substrate by a predisposed nanotube. The height of the nanotubular layer atop the substrate thus will be a multiple of the diameter of the nanotubes. Layers may be laid upon layers resulting in a sensor element whose height above the substrate is a plurality of the diameter of a nanotube, e.g., about 2, 3, 4, 5, 6,7, 8, 9, 10, 11, 12 or more. In some instances, an end of a nanotube may not be adhered to another molecule, but may be disposed at an angle pointing away from the substrate. These pointer nanotubes may thus have ends whose distances from the substrate later may exceed several diameters of the nanotubes. In an almost vertical disposition, the distance from substrate of one and of a nanotube may approach the length of the nanotube. It can be understood that nanotubes whose lengths are greater than the width are more likely to cross a predisposed nanotube. A stack two nanotubes in depth will present a maximum height off the substrate about two diameters of the nanotubes. But as subsequent layers are disposed nanotubes having increasing angles off the substrate will result in the sensor element height increasing in excess of the number of layers. An almost vertical nanotube will rise above the substrate, from a point of contact with the substrate or a nanotube in closer proximity to the substrate, to an elevation approximating the length of the nanotube off its contact height. The SWNT sensing layer thus grows in a stacked arrangement with carbon nanotubes forming a continuous solid phase interfused with gaps or holes allowing gases to convex and/or diffuse through the sensor element. While temperature is a common means for increasing kinetic energy of a molecule the designer, fabricator or operator of the inventive device may choose other methods for moving the molecules including, but not limited to: altered pressure differential, added vapor phase gas, transmitting vibration (including sonication), electromagnetic stimulation with visible or non-visible wavelengths of light (including laser stimulation), etc. Physical and/or electromagnetic stimulation increase temperature by increasing one or more molecule's kinetic energy; resistive heat coils, hotplates and liquid radiators are additional options that excite molecules upon contact for heating or reduce kinetic energy when chilling a sample. Any available source including, but not limited to: photo momentum, acoustic, gas flow, magnetic, electromagnet, electric, Lorentz force, chip replacement, etc., that excite or calm molecules upon contact can be used to modulate interaction of a compound, such as a VOC, and/or control delivery or movement of sample compounds.

Essentially flat and/or SWNT based sensors may be formed as lattice works, towers or spikes rising above the substrate's 2 dimensional surface to create a 3-dimensional architecture. The third dimension is formed, for example, by using controlled volume, depositions; inert, preferably removable filler frameworks; rutile-like or silk-like inclusions; crystalline and filament structures; or other methods to build up tower, random, or geometric structures.

The gases from most biological sources and many chemical synthetic processes include VOCs whose travels are modulated by attraction and/or in some cases repulsion especially when the sensor element is carrying a charge. The nanotechnology incorporated in such devices is suitable for desktop, wall mounted, handheld and other formats.

U.S. Pat. No. 8,609,025 to Johnson, hereby incorporated in its entirety by reference, includes teachings for making carbon tube nanosensors, including SWNTs with at least one nanotube directly adjacent to both a source and a drain electrode directly adjacent to an insulating layer that is directly disposed on a substrate. Johnson teaches that several polynucleotides having distinct sequences may be used to decorate or functionalize the sensing elements by adsorption on the nanotube surface. Also disclosed by Johnson, are artificial intelligence tools for analyzing the electric current (I) modulation that results from from molecular interaction with chemical substances delivered proximal to the sensors. Examples include those where an insulating layer is on a semiconducting layer which can be formed with appropriate doping. Semiconducting p-type nanotubes were selected using an ON/OFF gate voltage current ratio greater than 1000.

Johnson also describes a possible mechanism of sensor functioning involving the electrical conductivity of a SWNT FET device as sensitive to charged species bound near the nanotube sidewall when a molecule affects the local electric field near the conduction channel formed by the nanotube interacting in conjunction with neighbor nanotubes functionalized with one or more preselected adhered molecules, such as a single stranded polynucleotide.

Functionalizing molecules in the present invention may be hybrids, for example a nucleic acid portion hybridized with a peptide, a carbohydrate, a fatty acid, a phosphorous, sulfur, selenium moiety, etc. For example, peptide nucleic acids are one hybrid species involving a material with a single polynucleic acid covalently bound to at least one polypeptide or at least two strands of nucleic acids linked together by one or more peptide linkers. When the functional group binds or is in close proximity to a molecule, electrons are shifted within the functional group which then shifts the electric field in the carbon nanotube causing a change in the electrical properties,such as conductivity, current, etc., between the source and drain electrodes. In some embodiments, the substrate may be semiconducting.

However, the nanotube sidewall is preferably functionalized to render SWNT FETs useful as chemical sensors in accordance with this invention. Functionalization involves binding a desired molecular species to nanotubes. Functionalization features adding selected molecules to the nanotube surface thereby enhancing the sensitivity of the SWNT sensor element and by using different functionalizing molecules expands the range of molecules detectable by the SWNT-FET. Many classes of functionalization molecules may be used,.especially biopolymers when biological metabolites are being assayed. Oligonucleotides, sugars, lipids, polysaccharides, and and hybrids thereof are especially preferred. Polynucleotides are capable of adsorbing to the SWNT through the pi-pi stacking effects functionalizing using such molecules and/or hybrids thereof are especially preferred. Polynucleotides such as ssDNA are reversibly adsorbed to the SWNT to facilitate regeneration of the sensor. Particularly preferred polynucleotides display secondary structures that specifically bind to odor molecules. Most preferred is ssDNA that adsorbs on the SWNT and produces a positive electrostatic potential at the surface of the SWNT. Except where context indicates otherwise, the prefixes “oligo-” and “poly-” are used interchangeably.

U.S. Pat. No. 7,947,371, to Boussaad and Harmer, hereby incorporated in its entirety by reference, discusses methods for making nanotubes concentrating discussion on the difficulties involved in manipulating individual carbon nanotubes. Aggregation is particularly problematic, they say, because the highly polarized, smooth-sided fullerene tubes readily form parallel bundles or ropes with a large van der Woofs binding energy. This bundling amalgamates the electronic structures of the tubes to confound attempts to separate the tubes by size or type, or to use each as individual macromoiecular species. As a suggested solution, U.S. Pat. No. 7,498,423, to Zheng and Diner, issuing from US application 2004-0132072, teaches that “nucleic add molecules are able to singly disperse high concentrations of bundled carbon nanotubes in an aqueous solution.” (Boussaad and Harmer). However, Zheng and Diner report binding single stranded nucleic acid (ssDNA) to SWNTs in a manner avoiding functionalization. This was proposed as an improvement over previous methods (mentioned in their application paragraph [0005]). Novelty is described in paragraph [0007] as: “the present complexes are new, in that the association between the nanotube and nucleic add is non-covalent and not through the interaction of specific functionalized groups.”

In the present invention, a base layer of graphene, in an essentially fiat or in a nanotube format, may be deposited or grown, e.g., CVD onto a substrate, e.g., SiO₂/Si, The base layer may be immediately functionalized, but may be kept “as is” for subsequent nanotube stacking depositions which may be accomplished using nanotubes maintained in dispersion or dispersed in aqueous solution with nucleic add. The subsequent stacking layers may thus be applied using aqueous solutions with carbon nanotubes treated with one or more functionalizing groups such as polynucleic adds or other polymers. In other embodiments, a bare chemical sensor (a sensor having no biopolymer on a nanotube) can first be treated with a given DNA oligomer before a second biopolymer solution is applied to the DNA-decorated sensor.

As an alternative to a carbon nanotube base layer, the base layer may be two-dimensional, in the format of a non-tubular graphene. The construction is similar. Rather than constructing carbon nanotubes onto the substrate, graphene, either essentially flat or crumpled is disposed upon the substrate. Then subsequent layers of carbon nanotubes may be applied in a fashion similar to the application atop a ground layer comprising carbon nanotubes.

Nucleic add is typically applied to nanotubes, or agglomerations thereof, using an aqueous solution which includes buffers and salts under conditions that minimize nucleic acid association or hybridization with other nucleic adds. In such solutions, and in some cases with agitation or sonication, suspensions of agglomerated nanotubes release smaller and smaller agglomerations and eventually produce independent nanotubes with decorative nucleic add molecules adsorbed on theft surfaces. Different nucleic acids, e.g., different sequences, or different lengths may be used to functionalize the different sensor formats, graphene, based, 3-dimensional structures, and/or SWNT based.

The binding affinity of biopolymers, with ssDNA as an example, implies that there will be strong binding between the SWNT and the biopolymer. Molecules or volatile compounds bound by the biopolymers on the SWNT will be brought into close contact with the field effect sensor. This affords great compatibility with modern microfabrication techniques, the convenience of electronic readout, small footprint, and ease of fabrication. Useful biopolymers compatible with the present invention include, but are not limited to, polynucleotides such as DNA and RNA, polypeptides, nucleic acid-polypeptide complexes, carbohydrates, aptamers, ribozymes, and all homologs, analogs, conjugates, or derivatives thereof, as well as mixtures thereof. The term “polynucleotide,” generally refers to any polyribonucleotide or polydeoxribonucleotide, which may be unmodified RNA or DNA or modified RNA or DNA.

Polynucleotides include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide encompasses triple-stranded regions comprising RNA or DNA or both RNA and DNA. The term polynucleotide also includes DNAs or RNAs containing one or more modified bases and DNAs or RNAs with backbones modified for stability or for other reasons. Modified bases include, for example, tritylated bases and unusual bases such as inosine. A variety of modifications can be made to DNA and RNA; thus, polynucleotide embraces chemically, enzymatically or metabolically modified forms of polynucleotides as typically found in nature, as well as the chemical forms of DNA and RNA characteristic of viruses and cells. Polynucleotide, as used herein, also embraces relatively short nucleic acid chains, often referred to as oligonucleotides. Incorporating biopolymers onto the nanotubes of the present chemical sensors allows improved sensitivity and selectivity when multiple layers of nanotubes or disposed. This gives the device a gas-sensing functionality that utilizes individualized attraction or transient binding properties of differently decorated nanotubes to differentiate preferably select different VOCs. In preferred embodiments of the invention, single stranded DNA is used. These biopolymers provide a comprehensive library of compounds, each with specific binding characteristics

Biopolymers can be attached by reacting a solution of DNA with other biopolymers, such as RNA, peptides, etc., before application to a nanotube. A functionalizing solution may be applied to the primary nanotube layer before application of subsequent layers. The subsequent layers wherein solutions containing nanotubules are applied typically will be pre-functionalized. Functionalization is a simple method for preventing nanotube agglomeration. However, surfactants that do not adsorb as robustly to nanotubes in solution but that support their dispersion can be used in the deposition steps. The stacking disposition steps may vary in number depending on dispersant used (including functionalizing dispersants) and characteristic such as size of the nanotubes.

Selectivity of sensors is adjusted by doping or decorating the sensor framing or responsive molecule, e.g., an SWNT whose electronic perturbation affects electrical activity through the associated electrodes. In a typical example, single stranded oligonucleotides robustly pi-bond onto such SWNTs. These are preferred at present because they are easy to produce or obtain commercially and because they offer practically uncountable possibilities for doping. For example, oligonucleotides can be synthesized using nucleotides found in natural genetic materials, but non-natural nucleotide bases may easily be substituted, quickly compounding possibilities available for doping (functionalizing) and differentiating selectivities and sensitivities for assay devices. As an example, US Patent application 2010-0088040, by Johnson, provides a teaching used in the past to create SWNT sensors: “Single stranded oligonucleotides were obtained from Invitrogen (Carlsbad, Calif.) and diluted in distilled water to make a stock solution of 658 μg/ml (SEQ ID NO: 1) or 728 μg/ml (SEQ ID NO: 2). After odor responses of the bare SWNT-FET device were measured, a 500 μm diameter drop of ssDNA solution was applied to the device for 45 min, and then dried under a nitrogen stream. About 25 devices from two different SWNT growth runs were selected for detailed analysis and treated with ssDNA for the experiments.”

Nanotubes, preferably, SWNTs, may be arranged into devices that exhibit characteristics similar to conventional semiconductor field effect transistors (FETs). Embodiments using the sensors described herein are responsive to volatile components in gases where molecules in close proximity to the functionalized nanotubes disturb the electron distribution in the functionalizing compound which in turn leads o a current change in the associated FET. The responses differ in sign and magnitude for different VOCs, and for different arrangements of functional groupings on a sensor. Multiple nanotubes on a single sensor element especially in conjunction with a second, a third or more additional sensor elements with different sensation characteristics will detect molecules delivered to the sensing element in a sensor device. The time and strength of interaction can be recorded to detect subsequent interactions with the same or with similar compounds. In some embodiments, a bare chemical sensor (a sensor element having no biopolymer on its nanotube(s)) can first be treated with a given DNA oligomer, and if desired, a second biopolymer solution applied to the DNA-decorated sensor. Multiple biopolymers, e.g., oligos, may be disposed at different ratios to aid in differentiating VOCs that may preferentially interact with one or more of the disposed biopolymers.

A chip may be constructed to incorporate an arbitrary number of sensor elements. In practice, squares of numbers and/or powers or multiples of two or ten appear in many different embodiments, e.g., a dozen eggs, a six-pack of beer, a $10, $20, $50, $100 Federal Reserve Note, an 8-bit , 16-bit, 32-bit, 64-bit word, a kilobyte, megabyte, gigabyte, terabyte, etc. For purposes of example only, and not set out as a liming embodiment, a 256 SE (sensor element) chip is described in several configurations. SE chips might commonly include 10¹ SEs up to perhaps 10⁶ SEs. The size of the SE would allow for even greater numbers if desired. A practitioner may accordingly design a chip with an arbitrary number of SEs, e.g., about: 4 (2²), 10, 12, 15, 16 (24), 20, 24, 30, 32 (2⁵), 36, 50, 64 (26), 100 (^{˜}102), 128 (27), 256 (28), 512 (29), 1024 (210) (^{˜}103), 2048 (211), 4096 (212), 8192 (2¹³) (^{˜}10⁴), 16,384 (214), 32,768 (215), 65,536 (216), 131,072 (217) (^{˜}105), 262,144 (218), 524,288 (219), 1,048,576 (220) (^{˜}106), 2,097,152 (221), etc. A single SE chip may house a different decoration associated with each SE, may include multiple SEs with the same decorations, may include SEs with a combination of different decorations which may be decorated with different ratios of 2 or more functionalizing groups, may have rows, columns or individual chips at different base voltage or at different temperatures. Voltage and/or temperature maybe variably controlled between or during assay sessions. Variable controlling may be pre-programmed, may be manually input and/or may be algorithmically controlled in response to data collected.

A device may include an arbitrary multiple of SE chips which may be mounted on one or more cartridges, e.g., maybe up to 100 SE chips, for easy input if changeable features are desired. A cartridge may have SE chips of different capacities, e.g., two chips may feature four 16 SEs, four may feature 64 and another four may feature 256 SEs. The cartridge accordingly may carry an arbitrarily chosen number of chips, including, but not limited to: a single chip, 2 chips, 3, 4, 5, 6, 8, 10, 12, 15, 16, 20, 25, 36, 50, 64, 100, 128, 250, 256, etc. inclusive of numbers in between.

The device of the present invention provides rapid highly sensitive detection of VOCs in a gas phase sample. Sample data points are available on a continuous time basis. Instantaneous electronic measurements are digitized and stored and can be evaluated as averages over a selected time, a trend following a time, etc. Analytical data are then processed using the device's affiliated library of data and algorithms to detect a disease or to answer questions for which the sample was taken. As the device is used each or selected applications o(runs) of devices will continuously deliver additional data to the comparison libraries. Thus as the magnitude (volume) of use data increases the accuracy and specificity of diagnosis and suggested historically validated treatments increases. Through machine learning and artificial intelligence, the preferred device is continually developing and improving its algorithms.

A non-limiting example may present with an analytical device where a chip is built to have 32 SEs. Functionalizing groups may be different on all SEs on this chip. Accordingly, each SE will have a different set of functionalizing molecules. In other formats the chip may have 8 different functionalizing groups. Four SEs may share functionalizing groups. But the number of SEs with each functionalization is not required to be equal, e.g., 16 SEs may sport identical functionalizing groups, 8 may sport a different (second) functionalization, 6 may sport a third and 1 each may sport a fourth and fifth respectively. A similar consideration may be made with a different number, e.g., 4, 10, 12, 15, 16, 50, 64, 100, 128, 256, etc., of SEs on a chip. Each SE may have a functionalization different from all others on the chip. Or at least some SEs may share functionalizations with other SEs on the chip.

In the process of any analysis of the liquid samples and capturing the VOCs in vapor or gas phase to measure the presence, amounts, volume, intensity or strength of signal of multiple VOCs, then classifying each signal as from the organism or the environment and removing environmental (or foreign) VOCs from analysis consideration, the device can output a report of the sample's gross output of organism (or specific sample) initiated organic compounds. The report then formulates this gross output into a signature indicative of the sample. An associated data processor then compares the output signature to the signatures of the database to determine which VOCs are present and possibly to determine whether a specific disease (or set of diseases) may contribute to the signature.

The device comprises or has an associated computational component, said computational component programmed to accept input from at least a portion of said device, subject said input to analysis, and output a conclusion. The conclusion need not be a final conclusion but may, for example, be a set of instructions to refine further data collection. Instructions may be individualized wherein an instruction set may be determined for each SE. Where an instruction may be to ignore output from an SE, instruction to modulate its voltage, temperature or other feature, may be null. However, in some cases even though the output from a specific SE is not relevant, the temperature, voltage, etc., may be adjusted for effect such characteristics may have on neighboring SEs. For example, a set of SEs may be configured to attract one or more irrelevant compounds to reduce their interference with SEs whose detection data may be considered relevant.

Instructions sent to SEs may fluctuate during analysis of a single sample. For example, ramping temperature or other SE characteristic may optimize detection of different compounds at different mes (temperatures).

Ratios may be an important factor for generating a sample profile. Ratios may be altered during collection by changing the weighting given the various SEs. When outputs from multiple SEs are instrumental in identifying an quantifying a molecule, the ratio may be calculated using multiple SE outputs, including SE outputs from the same or from a different SE obtained at different mes during a sample analysis.

Methods relating to the present invention continue to consolidate VOC signature profiles into a growing library as each sample output is added.

At least one, but often a plurality of sensor chips, may be included in a device. During use, the sensor chips will be mounted in a controlled atmosphere chamber where vapor phase analyte will be introduced to contact with the sensor chips and thus the sensor elements. During analysis, input and output voltages are provided and monitored, respectively, as analyte is delivered to the ambient volume over the chip. Only a vapor phase analyte contacts the sensor elements in the portion formed with stacked nanotubes. This provides advantages over many liquid phase SWNT and similar sensors in that sensor size can be reduced without having to account for surface tension, liquid phase excipients are not necessary and turnover rate is not compromised by the requirement to remove the liquid carrier. In accordance with this invention a liquid phase assay capacity can be layered over the stacked nanotubule sensing formation.

The effect of multiple layering disposes nanotubes, preferably SWNTs, are pseudo-randomly disposed resulting in an inefficient packing arrangement at a first layer. The first layer has flat SWNTs. Since the subsequent layers can be at any angle, some may lay flat, but others will lie across at least one nanotube at a lower layer. This produces an unevenness with a three dimensional stacking with the nanotubes at angles along the surface, but also at angles from the surface. Occasional tubes will stand almost erect. The resulting effect is a porous structure where gas flow is impeded through the almost spongelike disposition of the molecule interacting surfaces. Thus a molecule softly impeded at a first near contact area is slowed and thus more likely to react with the next nanotubule. More attracted VOC molecules have the interacting events compounded with each close proximity event slowing the molecule and raising the probability of the next. These multiple interactions, on average, increase the number and the length of interactions. Thus differentiation between compounds of slightly different affinities is multiplied. Since the differences between reaction times and residence times in close proximity to a detecting surface in the sensor of even slightly differently attracted molecules are multiplied. Detection and identification are immensely improved.

In some embodiments, a sensor element may be topped off with a second insulating layer, in essence forming a sandwich of sensing SWNTs isolated top and bottom by a barricade. Graphene is an excellent isolator, impermeable even to most gases, but permeable to water vapor and smaller molecules. One or more isolation barricade(s) may be perforated or doped with impurity to permit controlled flow of gas through the perforations. The flow is controlled by the size of the perforations, the size of the diffusing or flowing gas molecule and a pressure differential across the insulating layer. Controlling doping or perforations gives a graphene layer a filtration function. Thus gas, which may include sample gas or a reactive gas such as water vapor may be added or subtracted to the sensing environment coincident with the sensing. A flat graphene may be progressively doped along one dimension to allow passage of different sized molecules along its length.

Samples may be introduced as desired by operator. For example a liquid sample may be provided to the device in a manner resulting in gases emitted from the liquid contact the SE. Heating a liquid sample can advantageously allow gases to evaporate off the liquid sample. Such heating can also provide a convective flow as the volume of gases expands. An inert gas, one non-reactive with samples, may be used to deliver gas vapors to the SE portions of the device. As temperatures increase the emission of some VOCs will be accelerated with respect to others. The elapsed time or temperature of the source liquid can be used as a data factor for identifying and quantifying the detected gases. Stability of gas compound may be an issue. Isomerization is one factor that is influenced by temperature, but a slower rate may occur at lower temperatures. So time itself, even when a sample is not heated can be a relevant data factor for characterizing and identifying detected compounds. Clearly, when the base voltage or temperature at an SE or a group of SEs time will elapse as the control factors are modified. Time may be used as a surrogate when the rate of change is well controlled.

A gas sample may be injected or otherwise inserted into the device for analysis. The gas may be collected from a headspace above a liquid, a steam or vapor driven from a liquid, a gas from any enclosed or ambient environment, a gas adsorbed upon and then driven from a collector. In embodiments where a graphene layer is built on the stacked nanotubes, perforations in the graphene either physically or by using dopants can serve to deliver the gas sample to the nanotube SEs. As with other liquid based sample provision systems, temperature or other molecular excitement may be employed to control and facilitate delivery of gas to the nanotube stacks. The gas may be allowed to diffuse in the device. Faster analyses will be achievable when the gas is delivered convectively, e.g., from a higher pressure to a reduced or lower pressure. Ambient or other sample gases may be remotely collected and stored, e.g., in an enclosed contained or adsorbed on a surface for later delivery to the device. The device may include a fan or pump for collecting or moving gases to access the SE regions.

Standardizing VOCs or other compounds may be used as controls to assist in determining quantities. A collection of standardizing VOCs of known electronic character and size may be used to characterize size of sample VOCs that may pass through the graphene barricade. One or more VOCs acting as standards may be present in the sample. For example, water vapor may be one parameter used as control to standardize concentration outputs.

The perforations may be of a size to allow vapors to cross while excluding liquid flows. In such formats, the system may be designed to assay larger molecules, i.e., molecules not effectively volatilized for assay in the gas phase, on an outer portion of the sandwich. The liquid assay layer may be configured in any sensing format, including, but not limited to ligands for targeted molecules, graphene—essentially flat, crumpled and/or tubular, enzymes, etc. In these formats, the device assays volatile gasses in the stacked portion of the sensing element and larger molecules delivered in liquid to the sandwich border. Such arrangement may be considered as two sensing elements where the liquid and gas phase portions deliver separate outputs or may be considered as a single element when the outputs from each layer are combined. Embodiments of the system are available where a liquid sample is disposed atop a sensing element construct for liquid carried molecules to be assayed on the peripheral surface while gases pass through for assaying volatile compounds below. The format does not require a vertical sandwich. The liquid phase may be introduced to contact an assay element in a horizontal setup or even a liquid may be fed through a pipe or tube surrounded by a liquid assay inner surface while gases vaporize through to the VOC assay layers.

Analysis may be programmed to proceed in a step-wise fashion. For example, outputs from a liquid phase assay may be used select subsequent procedures. For example, data from a liquid phase analysis may indicate a source of sample or a disease state whose information may be used to control other sensing elements, e.g., directing liquid or gas flow to a particular or a group of SE chips, adjusting a base voltage of one or more SEs, adjusting temperature, flow rate, concentrating a sample (e.g., by increasing pressure), diluting a sample generally, e.g., with a nonreactive or inert gas or with a selected additive, e.g.,water vapor, formic acid, etc.

Another example of a stepwise process may involve gas phase samples held in the assay (SEs) chamber to produce an initial data set. The initial set may be compared with VOC or other molecular data from an experiential database. The experiential database may show a match with A₁, A₂, A₃ and A₇. But no evidence of A₂₂, A₆, or C₃₂. While the sample gas remains in the chamber, either stationary or flowing through, the software of the device instructs as to the optimal additional diagnostic steps. For example, the gas may be diverted to a different part of a chip or may be channeled for analysis through another chip. Where the gas remains in the analysis zone of a chip, the software may tune one or more SEs, e.g., by increasing or decreasing temperature or voltage, weighting of output from a sensor in comparison to other sensors, and/or producing a pulsed voltage of a desired amplitude and frequency. The software associate with the device can use this data to eliminate refinements to detect and quantify compounds associated with A₂₂, A₆, and C₃₂, but not associated with A₁, A₂, A₃, or A₇, while adjusting the parameters of SEs associated with the additional compounds associated with A₁, A₂, A₃, or A₇. A second step where the adjusted SEs detect B₁₄, C₁₇, and not B₅ can eliminate B₅ associated conditions from consideration in diagnosis and any subsequent steps. The presence (and possibly ratios) of B₁₄ and C₁₇ may be sufficient to indicate a specific degenerative disease or pathogen associated with A₁, A₂, A₃, or A₇, B₁₄, and C₁₇, but not B5. This example only illustrates a potential of an interactive system that the device of this invention can be part of. The number of parameters can be much greater than the few used in the example and may not simply involve a determination of presence or absence but often will include quantities. Ratios of detected compounds to each other and/or to compounds serving as standards will often be key data points in the initial and subsequent analyses. Other types of devices may contribute to the experiential database. Such experiential database is not a required feature of the present invention.

As the device of the invention collects data, it may become part of a system using data from multiple devices to construct, maintain, and improve experiential libraries to continuously improve assay resources and refine the programmed methods for optimal reporting of the samples provenance including accurate suggestion of one or more disease states that may be associated with the sample(s).

One-, two-, or three- dimensional arrangements of the multiply layered sensors elements of the present invention may be incorporated into a device. Feeder gases may be flowed in an up or down manner, a cross manner, or a front to back manner with respect to an arbitrarily designated front of the machine. Flow direction may change during an analytical run either in a pre-programmed manner, under operator control or in accordance with one or more algorithms responsive to data collected during a given sample run and/or data collected from previous samples.

1. An analytical device comprising:
a substrate supporting:
a source electrode;
a drain electrode;
an insulating layer between said source and and said drain electrode;
a first graphene layer contacting said source and and said drain electrode to form a first sensing element;
a second graphene layer disposed on said first graphene layer, said second graphene layer consisting essentially of single wall carbon nanotubules (SWNTs) having a length and a diameter to form a second sensing element;
each said SWNT having a first end and a second end separated by said length, each said SWNT having a first distance between the closest point of said first end to said insulating layer and a second end having a second distance between the closest point of said second end to said insulating layer,
said SWNTs being disposed in a stacked formation, wherein a majority of said first distances and said second distances of individual SWNTs in said stacked formation have a difference at least one half of said SWNT diameter;
said first and said second sensing elements in electronic communication with a data processor that can process said data to form a profile;
said data processor comprising at least one output channel capable of delivering said profile to a processor capable of comparing said profile to a library of profiles, said library of profiles comprising profiles categorized by diagnosis.

2. The analytical device of claim 1, said library of profiles further comprises profiles categorized by treatment.

3. The analytical device of claim 1, wherein said first graphene layer comprises SWNTs.

4. The analytical device of claim 1, wherein said first graphene layer comprises essentially flat graphene.

5. The analytical device of claim 1, wherein said essentially flat graphene layer comprises crumpled graphene.

6. The analytical device of claim 1, wherein said second graphene layer comprises functionalized SWNTs.

7. The analytical device of claim 6, wherein said functionalized SWNTs are functionalized with an adsorbed biopolymer.

8. The analytical device of claim 7, wherein said biopolymer comprises a nucleic acid or oligonucleotide.

9. The analytical device of claim 8, wherein said nucleic acid or oligonucleotide comprises a DNA.

10. The analytical device of claim 9, wherein said nucleic acid or oligonucleotide comprises a ssDNA.

11. The analytical device of claim 8, wherein said nucleic acid or oligonucleotide comprises an RNA.

12. The analytical device of claim 1, wherein at least one of said first sensing elements and said second sensing element comprise at least two distinct functionalizing compounds.

13. The analytical device of claim 12 comprising a plurality of said first sensing elements, wherein a first member of said plurality and a second member of said plurality of said first sensing elements, comprise two distinct functionalizing compounds.

14. The analytical device of claim 12 comprising a plurality of said second sensing elements, wherein a first member of said plurality and a second member of said plurality of said second sensing elements comprise two distinct functionalizing compounds.

15. The analytical device of claim 1 further comprising a tertiary essentially flat graphene layer, said tertiary flat graphene layer separated from said first graphene layer by said second graphene layer.

16. The analytical device of claim 1 further comprising a second insulating layer, said second insulating layer separated from said first graphene layer by said second graphene layer.

17. The analytical device of claim 16 wherein said second insulating layer comprises gas porous perforations.

18. The analytical device of claim 17 wherein said second insulating layer comprises a barrier wherein gas pressure on the side of said second graphene layer facilitates flow of gas from from side of said second graphene layer to the other side of said barrier.

19. The analytical device of claim 1 wherein a majority of said first distances and said second distances of individual SWNTs in said stacked formation have a difference at least about equal to the SWNT diameter.

20. The analytical device of claim 1 wherein a majority of said first distances and said second distances of individual SWNTs in said stacked formation have a difference at least about twice the SWNT diameter.

21. The analytical device of claim 1 wherein a majority of said first distances and said second distances of individual SWNTs in said stacked formation have a difference at least about four times the SWNT diameter.

22. The analytical device of claim 15 wherein said tertiary flat layer is configured with sensing molecules for assaying molecules in a liquid carrier.

23. The analytical device of claim 22 wherein the tertiary flat layer comprises perforations through which vapors pass for analysis on a side opposite the side assaying molecules in a liquid layer.

24. The analytical device of claim 22 wherein the tertiary flat layer is associated with a molecular assay surface selected from the group consisting of: a ligand, a SWNT, and an enzyme.

25. The analytical device of claim 1 wherein at least one of said graphene layers is configured as a 3-dimensional structure rising above the substrate.

26. A method for selectively diagnosing a disease or condition, said method comprising:
obtaining a biosample from a subject; delivering liquid from said biosample to the analytical device of claim 1; collecting data outputted from said device to form a subject profile; comparing said subject profile to a library of profiles associated with one or more disease or condition, said library built from previous data outputs from said analytical device through an application of artificial intelligence; recognizing a positive comparison between said subject profile and a library profile associated with a disease; and reporting the associated disease or condition as a diagnosis.

27. A method for selecting a treatment for a disease or condition, said method comprising:
obtaining a biosample from a subject; delivering liquid from said biosample to the analytical device of claim 1; collecting data outputted from said device to form a subject profile; comparing said subject profile to a library of profiles associated with one or more treatments or conditions, said library built from previous data outputs from said analytical device through an application of artificial intelligence; recognizing positive comparisons between said subject profile and a library profile associated with said disease or condition; identifying treatments that produce best outcomes for profiles similar to said subject profile; and suggesting at least one of said treatments.

28. A method for determining efficacy of a treatment for a disease or condition reported in accord with claim 26, said method comprising: obtaining a second biosample from said subject; delivering liquid from said second biosample to the analytical device; collecting data outputted from said device to form a subject second profile; comparing said subject second profile to a library of profiles associated with one or more treatments or conditions, said library built from previous data outputs from said analytical device through an application of artificial intelligence; recognizing absence of a positive comparisons between said subject second profile and a library profile associated with said reported disease or condition; identifying said treatments as efficacious.

29. A method for improving efficacy of a treatment for a disease or condition reported in accord with claim 26, said method comprising: obtaining a second biosample from said subject; delivering liquid from said second biosample to the analytical device; collecting data outputted from said device to form a subject second profile; comparing said subject second profile to a library of profiles associated with one or more treatments or conditions, said library built from previous data outputs from said analytical device through an application of artificial intelligence; recognizing positive comparisons between said subject profile and a library profile associated with said disease or condition; identifying treatments that produce best outcomes for profiles similar to said subject second profile; and suggesting at least one of said treatments.
